(19) United States
(12) Patent Application Publication
(10) Pub. No.: US 2021/0375442 A1
(43) Pub. Date: Dec. 2, 2021

(54) PATIENT CARE EXCHANGE PORTAL WITH MARKET ANALYSIS

(71) Applicant: Duett, Inc., Indianapolis, IN (US)

(72) Inventors: Jonathan Rex Haag, Fishers, IN (US); Chad D. Bales, Indianapolis, IN (US)

(21) Appl. No.: 17/332,043

(22) Filed: May 27, 2021

Related U.S. Application Data
(60) Provisional application No. 63/033,537, filed on Jun. 2, 2020.

(51) Int. Cl. G16H 40/20; G06Q 10/06; G06Q 30/02; G06F 16/2457; G06F 16/955

(52) U.S. Cl. CPC G16H 40/20; G06Q 10/063112; G06F 16/955; G06Q 30/0201; G06F 16/2457; G06Q 10/06393

(57) ABSTRACT

Technologies for a patient care exchange portal with market analysis include a care exchange server and one or more care provider devices and care coordinator devices. The care exchange server registers multiple care providers and configures provider profiles. The care exchange server receives a care coordinator request and selects one or more care providers that match the request. The matching care providers are notified and may respond with interest to the request. The care coordinator and/or an individual patient may review the interested care providers and accept a care provider. The care exchange server may provide a personalized exchange view to the individual. The care exchange server may generate business intelligence and market analysis for a care provider. Other embodiments are described and claimed.

### CROSS-REFERENCE TO RELATED APPLICATION

This application claims priority under 35 U.S.C. § 119(e) to U.S. Provisional Patent Application No. 63/033,537, which was filed on Jun. 2, 2020. The above-referenced patent application is incorporated by reference in its entirety.

### BACKGROUND

In-home health care may include a wide range of health care services provided in home for illness or injury. Typically, individual patients and care coordinators may select a health care provider using a provider directory, which may include an overwhelming number of care providers. Such directories may be difficult to use and are often quickly out of date.

### SUMMARY

According to one aspect of the disclosure, a computing device for processing care requests includes a care provider manager, a data engine, a care coordinator dashboard, and a care provider dashboard. The care provider manager is to register a plurality of care providers. The care coordinator dashboard is to receive a care coordinator request indicative of one or more care attributes from a care coordinator. The data engine is to select a first set of the plurality of care providers matching the one or more care attributes. The care provider dashboard is to send the care coordinator request to the first set of the plurality of care providers, and receive an interest response from a second subset of the first set, wherein the interest response is indicative of interest in the care coordinator request. The care coordinator dashboard is further to provide the second subset of the plurality of care providers to the care coordinator, and receive an acceptance response from the care coordinator in response to provision of the second subset, wherein the acceptance response is indicative of an accepted care provider of the second subset. In an embodiment, the care attributes include a requested service, a requested location, and a requested funding source.

In an embodiment, the data engine is further to update a care provider reliability score associated with the accepted care provider in response to receiving the acceptance response. In an embodiment, to provide the second subset of the plurality of care providers to the care coordinator includes to provide a provider profile associated with each of the second subset, wherein the provider profile includes the reliability score. In an embodiment, to update the care provider reliability score includes to receive a reliability score from the care coordinator. In an embodiment, to update the care provider reliability score further includes to receive a reliability score from an individual patient associated with the care coordinator request.

In an embodiment, to provide the second subset of the plurality of care providers to the care coordinator includes to send a personalized exchange viewer to an individual patient associated with the care coordinator request. In an embodiment, the care coordinator dashboard is further to receive a negative acceptance response from the care coordinator in response to the provision of the second subset, wherein the negative acceptance response is indicative of a declined care provider of the second subset; and the care provider dashboard is further to send a notification to a third subset of the second subset in response to receipt of the negative acceptance response, wherein the third subset does not include the declined care provider.

In an embodiment, the computing device further includes a business intelligence engine to generate business intelligence data for a first care provider based on care provider data associated with the first care provider; and provide the business intelligence data to the first care provider. In an embodiment, the business intelligence data includes available care coordinator requests for a defined time period, reviewed care coordinator requests for the defined time period, or matched care coordinator requests for the defined time period.

In an embodiment, the computing device further includes a market analysis engine to receive a market analysis request including first care attributes from a first care provider; select care coordinator data that matches the first care attributes of the market analysis request; and provide the care coordinator data that matches the first care attributes to the first care provider. In an embodiment, the first care attributes include a care coordinator identity, a service offered, a funding source, or a service geography.

In an embodiment, to register the plurality of care providers includes, for each care provider, to configure a care provider profile, wherein the care provider profile includes services offered, service geography, or funding sources accepted.

In an embodiment, the computing device further includes a web portal that includes the care coordinator dashboard and the care provider dashboard.

According to another aspect, a method for processing care requests includes registering, by a care exchange server, a plurality of care providers; receiving, by the care exchange server, a care coordinator request indicative of one or more care attributes from a care coordinator; selecting, by the care exchange server, a first set of the plurality of care providers matching the one or more care attributes; sending, by the care exchange server, the care coordinator request to the first set of the plurality of care providers; receiving, by the care exchange server, an interest response from a second subset of the first set, wherein the interest response is indicative of interest in the care coordinator request; providing, by the care exchange server, the second subset of the plurality of care providers to the care coordinator; and receiving, by the care exchange server, an acceptance response from the care coordinator in response to providing the second subset, wherein the acceptance response is indicative of an accepted care provider of the second subset. In an embodiment, the care attributes include a requested service, a requested location, and a requested funding source.

In an embodiment, the method further includes updating, by the care exchange server, a care provider reliability score associated with the accepted care provider in response to receiving the acceptance response.

In an embodiment, providing the second subset of the plurality of care providers to the care coordinator includes sending a personalized exchange viewer to an individual patient associated with the care coordinator request.

In an embodiment, the method further includes generating, by the care exchange server, business intelligence data for a first care provider based on care provider data associated with the first care provider; and providing, by the care exchange server, the business intelligence data to the first care provider.

In an embodiment, the method further includes receiving, by the care exchange server, a market analysis request including first care attributes from a first care provider; selecting, by care exchange server, care coordinator data that matches the first care attributes of the market analysis request; and providing, by the care exchange server, the care coordinator data that matches the first care attributes to the first care provider.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for a patient care exchange portal with market analysis;

FIG. 2 is a simplified block diagram of at least one embodiment of an environment that may be established by a care exchange server of the system of FIG. 1;

FIG. 3 is a simplified state transition diagram of a care request process that may be executed by the system of FIG. 1;

FIGS. 4 and 5 are a simplified flow diagram of at least one embodiment of a method for processing care coordinator requests that may be executed by the care exchange server of FIGS. 1 and 2;

FIG. 6 is a simplified flow diagram of at least one embodiment of a method for generating business intelligence and market analysis that may be executed by the care exchange server of FIGS. 1 and 2;

FIG. 7 is a simplified flow diagram of at least one embodiment of a method for accessing the care exchange server that may be executed by a care provider device of the system of FIG. 1;

FIG. 8 is a simplified flow diagram of at least one embodiment of a method for accessing the care exchange server that may be executed by a care coordinator device of the system of FIG. 1;

FIG. 9 is a simplified flow diagram of at least one embodiment of a method for accessing a personalized care exchange viewer that may be executed by an individual patient device of the system of FIG. 1;

FIG. 10 is a schematic diagram of at least one embodiment of a care coordinator dashboard interface of the system of FIG. 1;

FIG. 11 is a schematic diagram of at least one embodiment of a coordinator request detail view interface of the system of FIG. 1;

FIG. 12 is a schematic diagram of at least one embodiment of a personalized exchange viewer interface of the system of FIG. 1;

FIG. 13 is a schematic diagram of at least one embodiment of a care provider dashboard interface of the system of FIG. 1;

FIG. 14 is a schematic diagram of at least one embodiment of a care provider request detail view interface of the system of FIG. 1; and

FIG. 15 is a schematic diagram of at least one embodiment of a market analysis interface of the system of FIG. 1.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); or (A, B, and C). Similarly, items listed in the form of “at least one of A, B, or C” can mean (A); (B); (C): (A and B); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

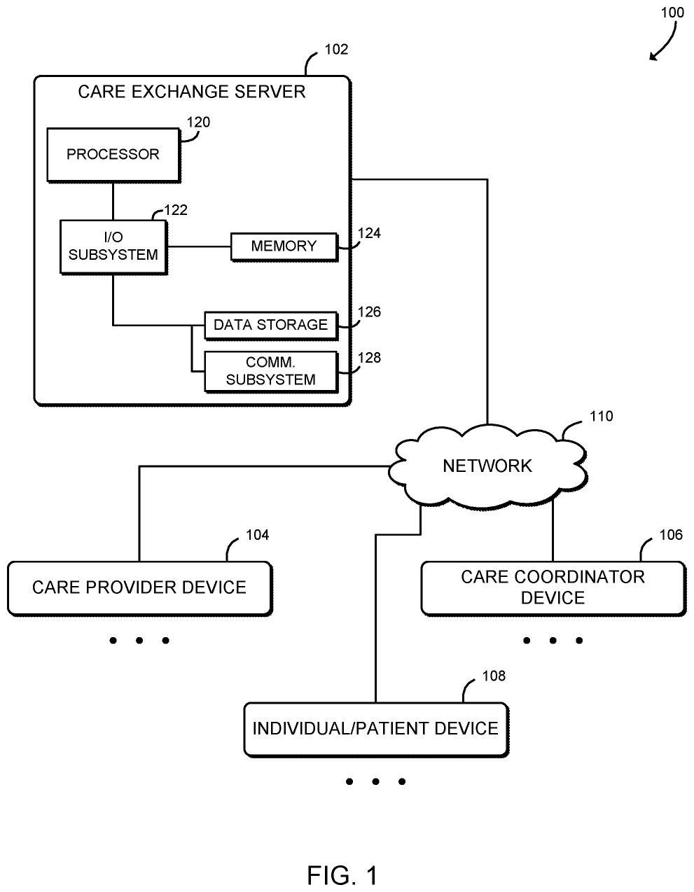

Referring now to FIG. 1, an illustrative system 100 for a patient care exchange with market analysis includes a care exchange server 102, multiple care provider devices 104, one or more care coordinator devices 106, and in some embodiments one or more individual/patient devices 108 in communication over a network 110. In use, a care coordinator device 106 issues a request for in-home health care to the care exchange server 102. The care exchange server 102 matches the request against a real-time database of care providers and sends the request to matching, eligible care provider devices 104. The care providers may express interest in serving requests to the care exchange server 102, and the care exchange server 102 presents those care providers to the care coordinator device 106 (and/or an individual device 108) for review. The care coordinator and/or the individual may accept a care provider. Additionally, care request data may be stored and analyzed in order to provide business intelligence and market analysis data to care provider devices 104. Accordingly, the system 100 allows individuals and coordinators to identify care providers based on real-time data on availability, geographic location, and other factors. Accordingly, the system 100 may improve the quality and/or efficiency of in-home health care delivery.

The care exchange server 102 may be embodied as any type of device capable of performing the functions described herein. For example, the care exchange server 102 may be embodied as, without limitation, a server, a rack-mounted server, a blade server, a workstation, a network appliance, a web appliance, a desktop computer, a laptop computer, a tablet computer, a smartphone, a consumer electronic device, a distributed computing system, a multiprocessor system, and/or any other computing device capable of performing the functions described herein. Additionally, in some embodiments, the care exchange server 102 may be embodied as a “virtual server” formed from multiple computing devices distributed across the network 110 and operating in a public or private cloud. Accordingly, although the care exchange server 102 is illustrated in FIG. 1 as embodied as a single computing device, it should be appreciated that the care exchange server 102 may be embodied as multiple devices cooperating together to facilitate the functionality described below. As shown in FIG. 1, the illustrative care exchange server 102 includes a processor 120, an I/O subsystem 122, memory 124, a data storage device 126, and communication circuitry 128. Of course, the care exchange server 102 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the care exchange server 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the care exchange server 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the care exchange server 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication circuitry 128 of the care exchange server 102 may be embodied as any communication circuit, device, or collection thereof, capable of enabling communications between the care exchange server 102, the care provider device 104, the care coordinator device 106, the individual/patient device 108, and/or other remote devices. The communication circuitry 128 may be configured to use any one or more communication technology (e.g., wireless or wired communications) and associated protocols (e.g., Ethernet, Bluetooth®, Wi-Fi®, WiMAX, etc.) to effect such communication.

Each care provider device 104, care coordinator device 106, and individual/patient device 108 is configured to access the care exchange server 102 and otherwise perform the functions described herein. Each of the care provider device 104, the care coordinator device 106, and the individual/patient device 108 may be embodied as any type of computation or computer device capable of performing the functions described herein, including, without limitation, a computer, a laptop computer, a notebook computer, a tablet computer, a wearable computing device, a multiprocessor system, a server, a rack-mounted server, a blade server, a network appliance, a web appliance, a distributed computing system, a processor-based system, and/or a consumer electronic device. Thus, each of the care provider device 104, the care coordinator device 106, and the individual/patient device 108 includes components and devices commonly found in a computer or similar computing device, such as a processor, an I/O subsystem, a memory, a data storage device, and/or communication circuitry. Those individual components of the care provider device 104, the care coordinator device 106, and the individual/patient device 108 may be similar to the corresponding components of the care exchange server 102, the description of which is applicable to the corresponding components of the care provider device 104, the care coordinator device 106, and the individual/patient device 108 and is not repeated herein so as not to obscure the present disclosure.

As discussed in more detail below, the care exchange server 102, the care provider device 104, the care coordinator devices 106, and the individual/patient device 108 may be configured to transmit and receive data with each other and/or other devices of the system 100 over the network 110. The network 110 may be embodied as any number of various wired and/or wireless networks. For example, the network 110 may be embodied as, or otherwise include, a wired or wireless local area network (LAN), a wired or wireless wide area network (WAN), and/or a publicly-accessible, global network such as the Internet. As such, the network 110 may include any number of additional devices, such as additional computers, routers, and switches, to facilitate communications among the devices of the system 100.

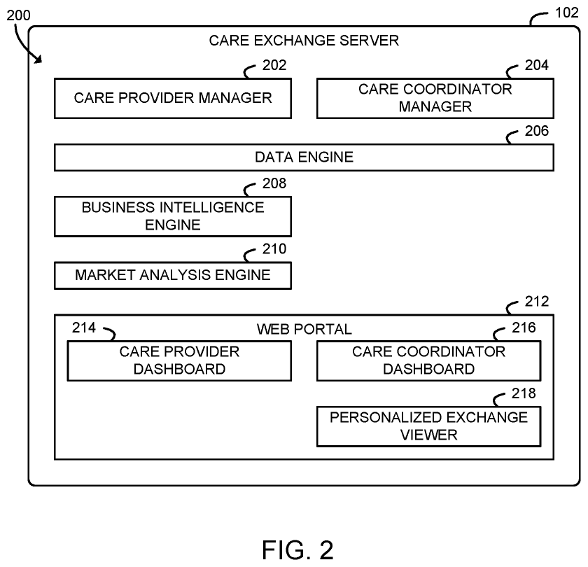

Referring now to FIG. 2, in the illustrative embodiment, the care exchange server 102 establishes an environment 200 during operation. The illustrative environment 200 includes a care provider manager 202, a care coordinator manager 204, a data engine 206, a business intelligence engine 208, a market analysis engine 212, and a web portal 212. The web portal further includes a care provider dashboard 214, a care coordinator dashboard 216, and a personalized exchange viewer 218. The various components of the environment 200 may be embodied as hardware, firmware, software, or a combination thereof. As such, in some embodiments, one or more of the components of the environment 200 may be embodied as circuitry or a collection of electrical devices (e.g., care provider manager circuitry 202, care coordinator manager circuitry 204, data engine circuitry 206, business intelligence engine circuitry 208, market analysis engine circuitry 212, and/or web portal circuitry 212). It should be appreciated that, in such embodiments, one or more of those components may form a portion of the processor 120, the I/O subsystem 122, and/or other components of the care exchange server 102.

The care provider manager 202 is configured to register one or more care providers. Registering the plurality of care providers may include, for each care provider, configuring a care provider profile, including services offered, service geography, or funding sources accepted. The care coordinator manager 204 is configured to register one or more care coordinators.

The care coordinator dashboard 216 is configured to receive a care coordinator request indicative of one or more care attributes from a care coordinator. The care attributes may include a requested service, a requested location, and a requested funding source.

The data engine 206 is configured to select an eligible set of the plurality of care providers that match the one or more care attributes. The data engine 206 is further configured to update a care provider reliability score associated with a care provider when the request is accepted as described further below. Updating the care provider reliability score may include receiving a reliability score from the care coordinator and/or receiving a reliability score from an individual patient associated with the care coordinator request.

The care provider dashboard 214 is configured to send the care coordinator request to the eligible set of the plurality of care provider in response to selection of the eligible set, and to receive an interest response from an interested subset of the eligible set, wherein the interest response is indicative of interest in the care coordinator request

The care coordinator dashboard 216 is further configured to provide the interested subset of the care providers to the care coordinator, and to receive an acceptance response from the care coordinator. The acceptance response is indicative of an accepted care provider of the interested subset. The care coordinator dashboard 216 may be further configured to send a personalized exchange viewer to an individual patient associated with the care coordinator request. The personalized exchange viewer 218 is configured to provide the interested subset of the plurality of care providers to the individual patient, and to receive an acceptance response from the individual.

The business intelligence engine 208 is configured to generate business intelligence data for a care provider based on care provider data associated with that care provider, and to provide the business intelligence data to the associated care provider. Business intelligence data may include available care coordinator requests for a defined time period, reviewed care coordinator requests for the defined time period, or matched care coordinator requests for the defined time period.

The market analysis engine 212 is configured to receive a market analysis request including care attributes from a care provider, to select care coordinator data that matches those care attributes, and to provide the care coordinator data that matches the first care attributes to the associated care provider. The care attributes may include a care coordinator identity, a service offered, a funding source, or a service geography.

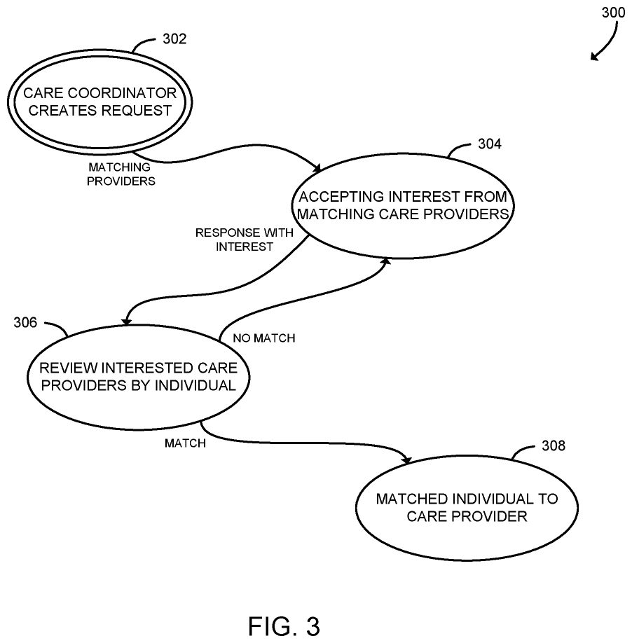

Referring now to FIG. 3, state transition diagram 300 illustrates the lifecycle of a care coordinator request processed by the system 100. The request starts in state 302, in which a care coordinator creates a request for care. The request specifies one or more attributes that define the requested care. The request transitions to state 304, in which care providers that match the request may express interest in the care coordinator request. The request may remain in the accepting state 304 for a preconfigured timeframe. In response to an expression of interest by one or more care providers, the request transitions to state 306, in which the care coordinator and/or the individual patient reviews the interested care providers and determines whether to match with the care provider. If no match occurs, the request transitions back to the accepting state 304, in which other care providers again have the opportunity to express interest. If a match occurs, the request transitions to state 308, in which the individual patient is matched to the care provider.

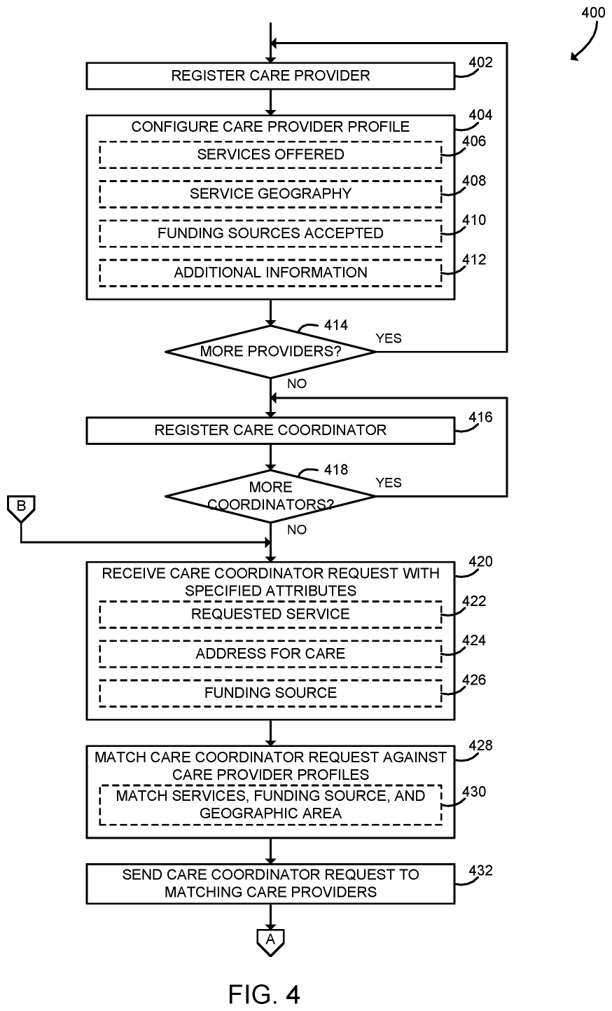

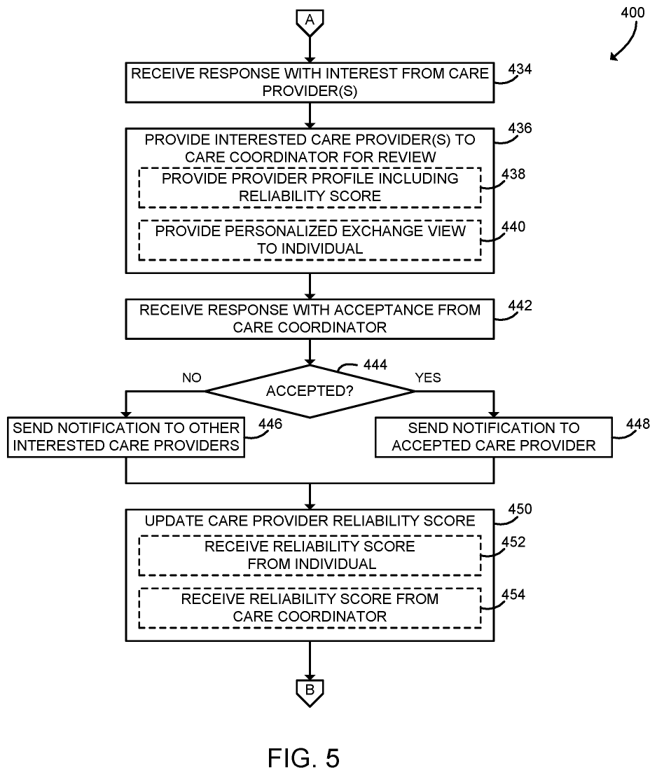

Referring now to FIGS. 4 and 5, in use, the care exchange server 102 may execute a method 400 for processing care coordinator requests. It should be appreciated that, in some embodiments, the operations of the method 400 may be performed by one or more components of the environment 200 of the care exchange server 102 as shown in FIG. 2. The method 400 begins with block 402, in which the care exchange server 102 registers a care provider. The care exchange server 102 may, for example, set up an account for the care provider or otherwise grant one or more care provider devices 104 access to the web portal 212 of the care exchange server 102.

In block 404, the care exchange server 102 configures a care provider profile associated with the care provider. The care provider profile includes information associated with the care provider and services offered. The care exchange server 102 may configure the care provider profile for a newly registered care provider or may update an existing care provider profile. In block 406, the care exchange server 102 may update services offered by the care provider, including types of service and availability. In block 408, the care exchange server 102 may update a service geography of the care provider. The service geography may include geographic regions (e.g., neighborhoods, postal codes, census tracts, political subdivisions, or other geographic regions) in which the care provider offers services. In block 410, the care exchange server 102 may update one or more funding sources accepted by the care provider. In block 412, the care exchange server 102 may update additional information in the care provider profile, such as a short marketing profile for the care provider or contact information for the care provider (e.g., website address, email address, phone number, or other contact information).

Although illustrated as being performed sequentially, it should be understood that the care exchange server 102 may update provider profile information at any time, and thus the provider profile information may reflect the real-time status of the care provider. In block 414, the care exchange server 102 determines whether to register additional care providers. If so, the method 400 loops back to block 402 to continue registering additional care providers. If the care exchange server 102 determines not to register any additional care providers, the method 400 advances to block 416.

In block 416, the care exchange server 102 registers a care coordinator. The care exchange server 102 may, for example, set up an account for the care coordinator or otherwise grant one or more care coordinator devices 106 access to the web portal 212 of the care exchange server 102. In block 418, the care exchange server 102 determines whether to register additional care coordinators. If so, the method 400 loops back to block 416 to continue registering additional care coordinators. If the care exchange server 102 determines not to register any additional care coordinators, the method 400 advances to block 420.

In block 420, the care exchange server 102 receives a care coordinator request with one or more specified care attributes. The care coordinator request is originated by a care coordinator (e.g., with a care coordinator device 106) and represents a request for in-home care associated with an individual patient. The care attributes may include one or more requirements for matching a care provider with the request. The care coordinator request may be submitted, for example, from the care coordinator device 106 via the care coordinator dashboard 216 of the web portal 212. In some embodiments, in block 422 the care attributes may include a requested service. In some embodiments, in block 424 the care attributes may include an address for the requested care (e.g., a home address for the individual). In some embodiments, in block 426 the care attributes may include a funding source.

In block 428, the care exchange server 102 matches the care coordinator request against the registered care provider profiles to identify one or more matching care providers. The care exchange server 102 may, for example, identify care providers that match some or all of the care attributes supplied with the care coordinator request. In some embodiments, in block 430 the care exchange server 102 may match the services provided by the care provider against the requested services, the funding sources accepted by the care provider against the requested funding source, and the geographic area serviced by the care provider against the requested address for care. Matching care providers may match all of those attributes (e.g., matching all of service, funding source, and geographic area).

In block 432, the care exchange server 102 sends the care coordinator request to all matching care providers. For example, the care exchange server 102 may add the care coordinator request to the care provider dashboard 214 of the web portal 212, thus allowing matching care provider devices 104 to access the care coordinator request.

In block 434, shown in FIG. 5, the care exchange server 102 receives a response indicating interest in the care coordinator request from one or more of the care providers. For example, the care exchange server 102 may receive the response with interest from a care coordinator device 104 via the care provider dashboard 214 of the web portal 212.

In block 436, the care exchange server 102 provides the interested care providers to the care coordinator for review. The care exchange server 102 may, for example, provide access to data from the associated care provider profile to the care coordinator device 106 via the care coordinator dashboard 216 of the web portal 212. In some embodiments, in block 438 the care exchange server 102 may provide a provider profile including a reliability score. The reliability score may be a percentage or other numerical score indicating reported reliability of the care provider. The reliability score may be determined based on information received from the care coordinator and from the individual patient. In some embodiments, in block 440 the care exchange server 102 may provide a personalized exchange view to the individual patient. For example, the care exchange server 102 may send an email message or other communication to the associated individual device 108 that includes a web address (e.g., URL) for the personalized exchange viewer. The personalized exchange view may include a summary of the coordinator request, provider profile information for the interested care providers, and controls to allow the individual to select one of the care providers.

In block 442, the care exchange server 102 receives a response that includes an indication of acceptance from the care coordinator. The response may indicate whether the patient accepted (matched) one of the interested care providers, and may identify the matched care provider. The response may be received, for example, from the care coordinator device 106 via the care coordinator dashboard 216 or from the individual device 108 via the personalized exchange viewer 218.

In block 444, the care exchange server 102 determines whether a care provider was accepted. If not, the method 400 branches to block 446, in which the care exchange server 102 sends a notification to other interested care providers that the care coordinator request has not been accepted. After sending the notification, the method 400 proceeds to block 450, described below. Referring back to block 444, if the care exchange server 102 determines that a care provider has been accepted, the method 400 branches to block 448, in which the care exchange server 102 sends a notification to the accepted care provider. After sending the notification, the method 400 advances to block 450.

In block 450, the care exchange server 102 updates the care provider reliability score associated with the accepted care provider. As described above, the reliability score may be a percentage or other numerical score indicating reported reliability of the care provider. In some embodiments, in block 452 the care exchange server 102 may receive a reliability score from the individual, for example from the individual device 108. In some embodiments, in block 454, the care exchange server 102 may receive a reliability score from the care coordinator, for example from the care coordinator device 106. After updating the care provider reliability score, the method 400 loops back to block 420, shown in FIG. 4, to continue processing care coordinator requests.

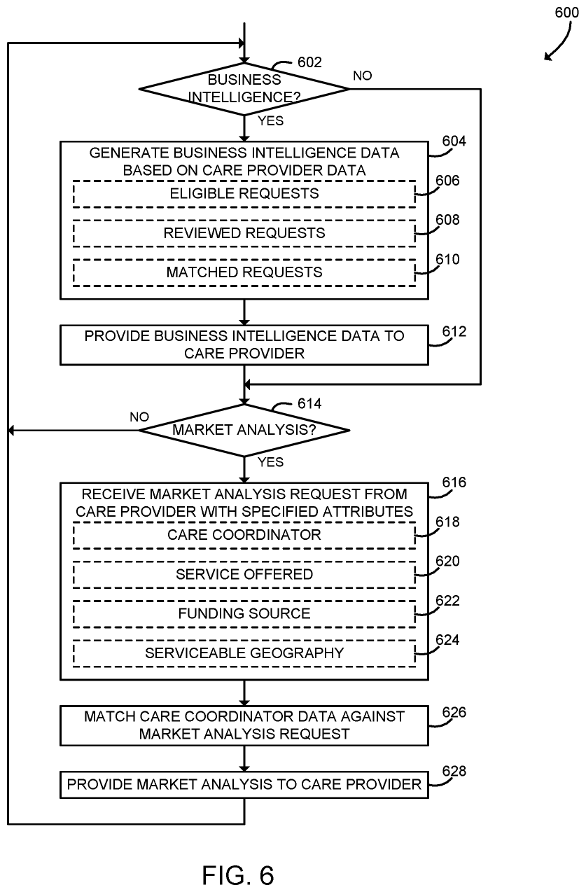

Referring now to FIG. 6, in use, the care exchange server 102 may execute a method 600 for generating business intelligence and market analysis. It should be appreciated that, in some embodiments, the operations of the method 600 may be performed by one or more components of the environment 200 of the care exchange server 102 as shown in FIG. 2. The method 600 begins with block 602, in which the care exchange server 102 determines whether to generate business intelligence for a care provider. Business intelligence may be included, for example, via the care provider 214 of the web portal 212. If the care exchange server 102 determines not to provide business intelligence, the method 600 branches ahead to block 614, described below. If the care exchange server 102 determines to generate business intelligence, the method 600 advances to block 604.

In block 604, the care exchange server 102 generates business intelligence data based on historical data for a particular care provider. The data may be recorded, for example, in response to processing care coordinator requests as described above in connection with FIGS. 4-5. The business intelligence data may be limited to a selected time period, for example within the last seven days, within the last month, or otherwise. In some embodiments, in block 606 the care exchange server 102 may determine a number of care coordinator requests for which the care provider was eligible. For example, the eligible care provider requests may be determined based on care coordinator requests with matching care attributes. In some embodiments, in block 608 the care exchange server 102 may determine a number of care coordinator requests that were submitted for review by the care coordinator (i.e., for which the care provider expressed interest). In some embodiments, in block 610 the care exchange server 102 may determine a number of matched care coordinator requests (i.e., for which the care coordinator and/or individual patient accepted the care provider). In block 612, the care exchange server 102 provides the generated business intelligence data to the care provider. The business intelligence data may be sent to the care provider device 104 for example via the care provider dashboard 214 of the web portal 212.

In block 614, the care exchange server 102 determines whether to perform a market analysis. The care exchange server 102 may perform market analysis, for example, in response to a command or other selection received from the care provider via the care provider dashboard 214. If the care exchange server 102 determines not to perform a market analysis, the method 600 loops back to block 602. If the care exchange server 102 determines to perform market analysis, the method 600 advances to block 616.

In block 616, the care exchange server 102 receives a market analysis request from the care provider that includes one or more specified care attributes. The care attributes may include one or more requirements for matching care requests. The market analysis request may be submitted, for example, from the care provider device 104 via the care provider dashboard 214 of the web portal 212. In some embodiments, in block 618 the market analysis request may specify a particular care coordinator. In some embodiments, in block 620 the market analysis request may specify one or more offered services. In some embodiments, in block 622 the market analysis request may specify one or more funding sources. In some embodiments, in block 624 the market analysis request may specify a serviceable geography.

In block 626, the care exchange server 102 matches care coordinator data against the market analysis request. The care exchange server 102 may identify data from care coordinator requests that match some or all of the specified care attributes of the market analysis request. The care coordinator data may be stored, for example, during processing of care coordinator requests as described above in connection with FIGS. 4-5. In block 628, the care exchange server 102 provides the market analysis data to the care provider. The data may be provided in graphical form, in tabular form, or in any other appropriate form for data presentation and visualization. For example, the care provider device 106 may access the market analysis data via the care provider dashboard 214 of the web portal 212. After providing the market analysis data, the method 600 loops back to block 602 to continue providing business intelligence and/or market analysis.

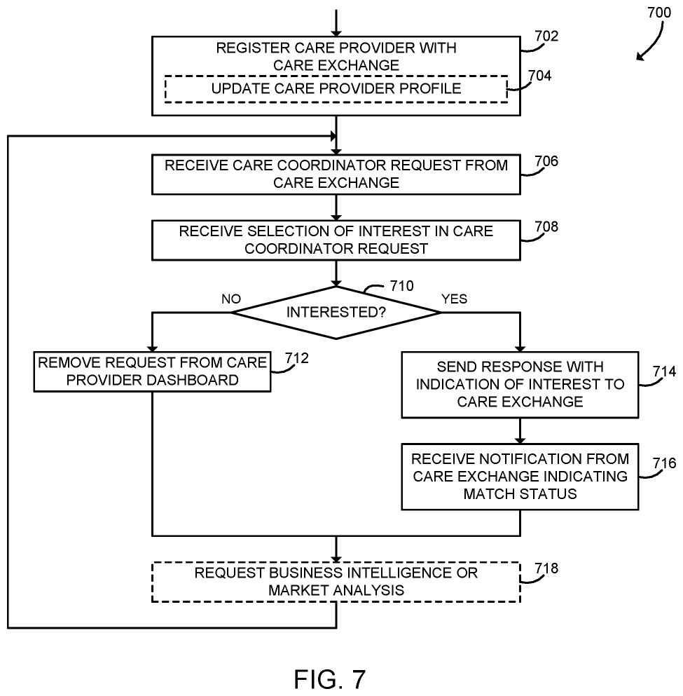

Referring now to FIG. 7, in use, a care provider device 104 may execute a method 700 for accessing the care exchange server 102. It should be appreciated that, in some embodiments, the operations of the method 700 may be performed by accessing one or more components of the environment 200 of the care exchange server 102 as shown in FIG. 2, such as the care provider dashboard 214. For example, the care provider device 104 may submit web requests to the care exchange server 102, receive web pages generated by the care exchange server 102, and otherwise communicate with the care exchange server 102. The method 700 begins in block 702, in which the care provider device 104 registers a care provider with the care exchange server 102. In some embodiments, in block 704 the care provider device 104 may update a care provider profile associated with the care provider. As described above, the care provider profile includes information associated with the care provider and services offered, including services offered, service geography, funding sources accepted, and other information such as marketing profile and contact information.

In block 706, the care provider device 104 receives a care coordinator request from the care exchange server 102. As described above, the care coordinator request is originated by a care coordinator and represents a request for in-home care associated with an individual patient. The care provider device 104 may receive only care coordinator requests for which the care coordinator is eligible (e.g., with matching requested service, geography, and funding source).

In block 708, the care provider device 104 receives a selection of interest in the care coordinator request. The selection indicates whether the care provider is interested in servicing the associated care coordinator request. The selection may be received, for example, via a user interface of the care provider device 104. In block 710, the care provider device 104 checks whether the care provider is interested. If not, the method 700 branches to block 712, in which the care provider device 104 removes the care coordinator request from the care provider dashboard 214. After removing the request, the method 700 may advance to block 718. In some embodiments, the method 700 may loop back to block 706 to process additional care coordinator requests.

Referring back to block 710, if the care provider is interested in servicing the care coordinator request, the method 700 branches to block 714, in which the care provider device 104 sends a response with an indication of interest to the care exchange server 102. As described above, after sending the response with indication of interest, the care provider is submitted to the care coordinator for review. In block 716, the care provider device 104 receives a notification from the care exchange server 102 that indicates match status. The notification may, for example, indicate whether or not the care provider was accepted by the care coordinator. After receiving the notification, the method 700 may advance to block 718. In some embodiments, the method 700 may loop back to block 706 to process additional care coordinator requests.

In block 718, the care provider device 104 requests business intelligence and/or market analysis from the care exchange server 102. As described above, the market analysis request may include one or more specified care attributes, such as care coordinator, service, funding source, geography, or other attributes. The care provider device 104 receives the requested business intelligence and/or market analysis from the care exchange server 102. After requesting the business intelligence and/or market analysis, the method 700 loops back to block 706 to process additional care coordinator requests.

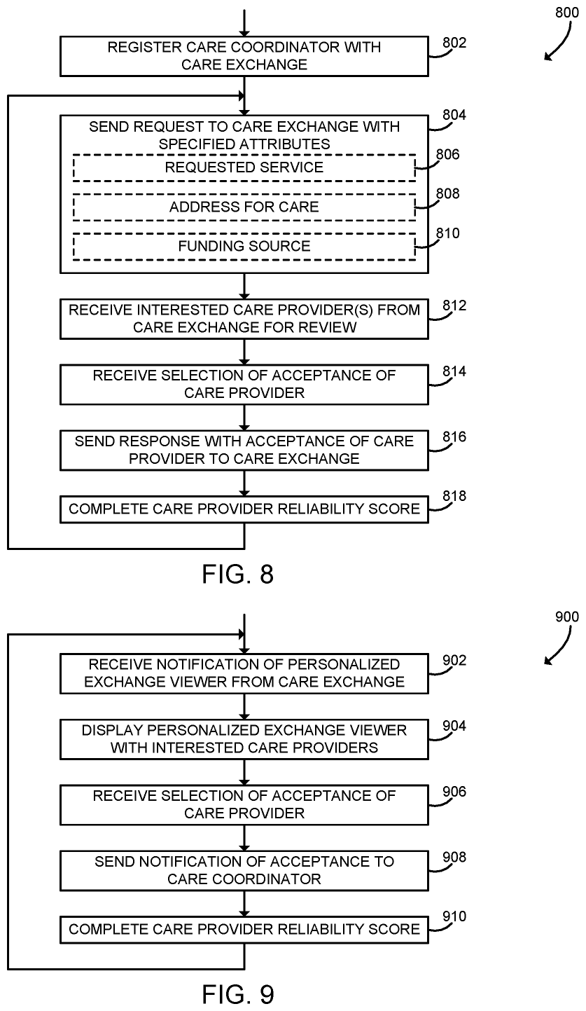

Referring now to FIG. 8, in use, a care coordinator device 106 may execute a method 800 for accessing the care exchange server 102. It should be appreciated that, in some embodiments, the operations of the method 800 may be performed by accessing one or more components of the environment 200 of the care exchange server 102 as shown in FIG. 2, such as the care coordinator dashboard 216. For example, the care coordinator device 106 may submit web requests to the care exchange server 102, receive web pages generated by the care exchange server 102, and otherwise communicate with the care exchange server 102. The method 800 begins in block 802, in which the care coordinator device 106 registers a care coordinator with the care exchange server 102.

In block 804, the care coordinator device 106 sends a care coordinator request with one or more specified care attributes to the care exchange server 102. As described above, the care coordinator request represents a request for in-home care associated with an individual patient. The care attributes may include one or more requirements for matching a care provider with the request. In some embodiments, in block 806 the care attributes may include a requested service. In some embodiments, in block 808 the care attributes may include an address for the requested care (e.g., a home address for the individual). In some embodiments, in block 810 the care attributes may include a funding source.

In block 812, the care coordinator device 106 receives one or more interested care providers for review from the care exchange server 102. As described above, the interested care providers are those care providers that are eligible for the care coordinator request (e.g., with matching requested service, geography, and funding source) and have expressed interest to the care coordinator request. The care providers may be reviewed by the care coordinator and/or by the individual patient. For example, the patient may review the care provider using a personalized exchange viewer as described below in connection with FIG. 9.

In block 814, the care coordinator device 106 receives a selection of acceptance of one of the interested care providers. The acceptance indicates whether an individual has accepted (matched) a particular care provider. The selection may be received, for example, via a user interface of the care coordinator device 106, or via the individual patient device 108. In block 816, the care coordinator device 106 sends a response including the acceptance of the care provider to the care exchange server 102. In block 818, the care coordinator device 106 completes a care provider reliability score. The care coordinator device 106 may send a score or other information indicative of reliability of the accepted care provider to the care exchange server 102. After completing the reliability score, the method 800 loops back to block 804 to continue processing additional care coordinator requests.

Referring now to FIG. 9, in use, an individual device 108 may execute a method 900 for accessing a personalized care exchange viewer. It should be appreciated that, in some embodiments, the operations of the method 900 may be performed by accessing one or more components of the environment 200 of the care exchange server 102 as shown in FIG. 2, such as the personalized exchange viewer 218. For example, the individual device 108 may submit web requests to the care exchange server 102, receive web pages generated by the care exchange server 102, and otherwise communicate with the care exchange server 102. The method 900 begins in block 902, in which the individual device 108 receives a notification of a personalized exchange viewer from the care exchange server 102. For example, the individual device 108 may receive an email message or other communication from includes a web address (e.g., URL) for the personalized exchange viewer provided by the care exchange server 102.

In block 904, the individual device 108 displays the personalized exchange viewer including interested care providers received from the care exchange server 102. As described above, the interested care providers are those care providers that are eligible for the care coordinator request (e.g., with matching requested service, geography, and funding source) and have expressed interest to the care coordinator request. The care providers may be reviewed by the individual patient. In block 906, the individual device 108 receives a selection of acceptance of one of the interested care providers. As described above, the acceptance indicates whether the individual has accepted (matched) a particular care provider. The selection may be received, for example, via a user interface of the individual device 108. In block 908, the individual device 108 sends a response including the acceptance of the care provider to the care coordinator device 106 and/or to the care exchange server 102. In block 910, the individual device 108 completes a care provider reliability score. The individual device 108 may send a score or other information indicative of reliability of the accepted care provider to the care exchange server 102. After completing the reliability score, the method 900 loops back to block 902, in which the individual device 102 may continue to access the personalized care exchange viewer.

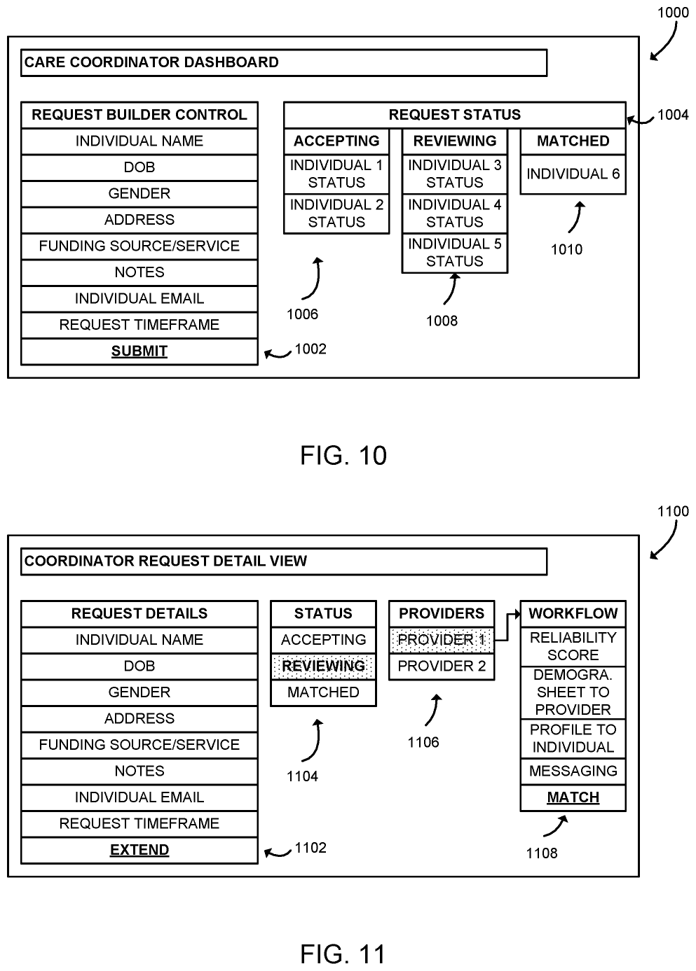

Referring now to FIG. 10, user interface 1000 illustrates one potential embodiment of a user interface of the care coordinator dashboard 216. The user interface 1000 may be embodied as a web page, native application, or other interface provided by the care coordinator device 106 for interaction with the care exchange server 102. The user interface 1000 illustratively includes a request builder control 1002, which may be used to configure the care attributes of a care coordinator request and to submit the request to the care exchange server 102. The user interface 1000 further includes a request status control 1004, which displays information for pending care coordinator requests organized by status. An accepting list 1006 displays requests that are currently accepting interest from matching care providers. A reviewing list 1008 displays requests that have received interest from care providers and may be reviewed by the care coordinator and/or the individual patient. A matched list 1010 displays requests that have been accepted (matched) by the care coordinator and/or the individual patient. Each entry in the request status control 1004 may include summary information (e.g., patient name), and when selected may provide additional details as shown in FIG. 11, described below.

Referring now to FIG. 11, user interface 1100 illustrates one potential embodiment of a coordinator request detail view user interface of the care coordinator dashboard 216. The user interface 1100 may be embodied as a web page, native application, or other interface provided by the care coordinator device 106 for interaction with the care exchange server 102. The user interface 1100 includes a request detail control 1102, which displays care attributes of a selected care coordinator request, including the request timeframe. The illustrative request detail control 1102 also includes a control to extend the request timeframe (e.g., if sufficient care providers have not yet expressed interest). A request status control 1104 indicates the status of the selected request, which is illustratively “reviewing” (i.e., being reviewed by the care coordinator and/or the individual patient). A provider control 1106 lists providers that have expressed interest in the care coordinator request. Each provider in the provider control 1106 is selectable, and when a provider is selected, the workflow control 1108 associated with that provider is active. For example, as shown in FIG. 11, the provider 1 is illustratively selected. The workflow control 1108 includes data and/or actions related to the selected care provider. The illustrative workflow control 1108 includes controls and/or links to view reliability score of the care provider, send a demographic sheet to the provider, send a provider profile to the individual (e.g., by sending a link to the personalized exchange viewer), send/receive messages with the provider, and match (i.e., accept) the provider.

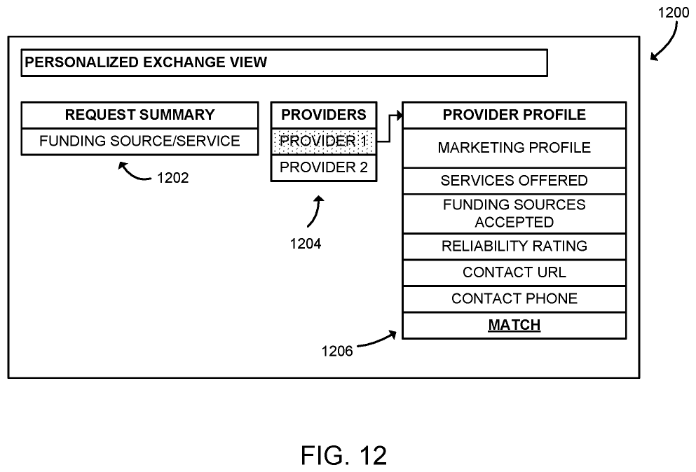

Referring now to FIG. 12, user interface 1200 illustrates one potential embodiment of a coordinator request detail view user interface of the personalized exchange viewer 218. The user interface 1200 may be embodied as a web page, native application, or other interface provided by the individual device 108 for interaction with the care exchange server 102. The user interface 1200 includes a request summary control 1202, which displays certain selected attributes of a care coordinator request. A provider control 1204 lists providers that have expressed interest in the care coordinator request. Each provider in the provider control 1204 is selectable, and when a provider is selected, the provider profile control 1206 associated with that provider is active. The provider profile control 1206 includes information related to the selected provider, including provider profile data, the reliability rating, and other data. The provider profile control 1206 also includes a control to match (i.e., accept) the provider.

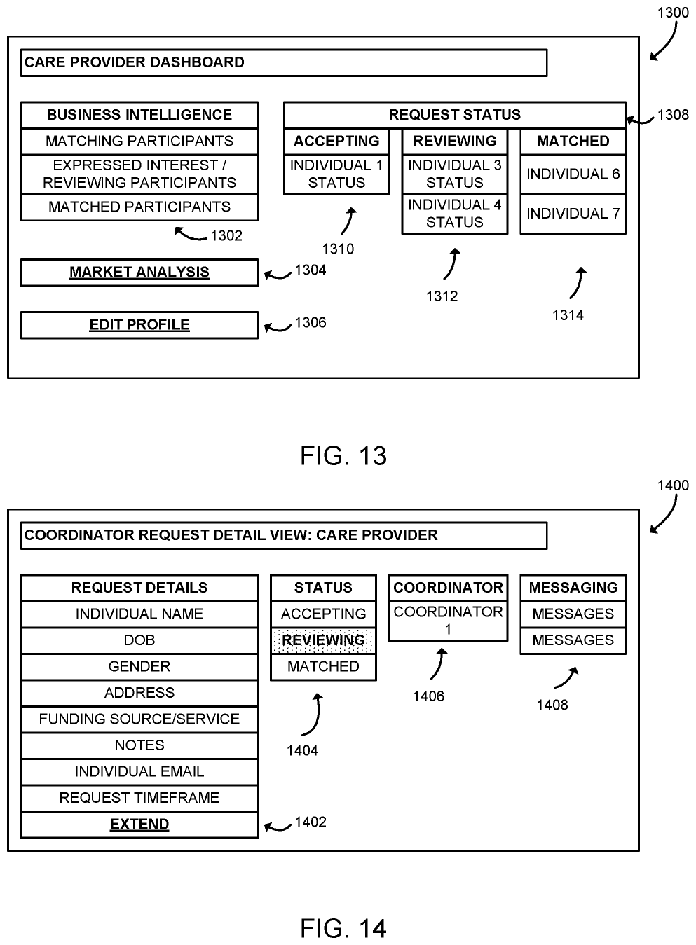

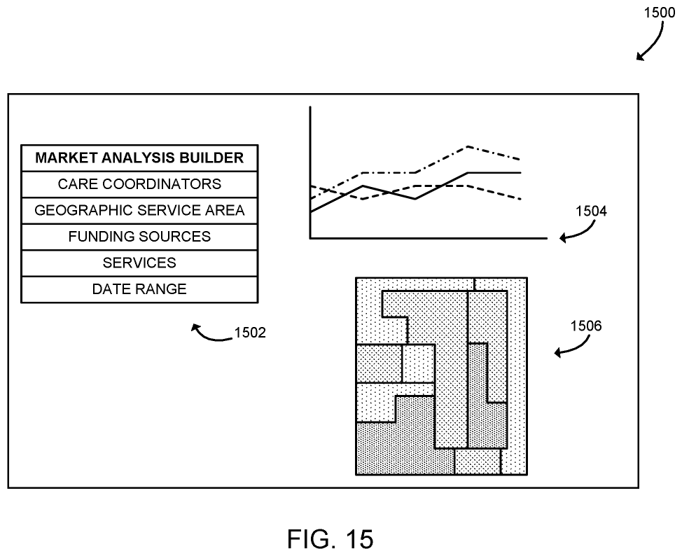

Referring now to FIG. 13, user interface 1300 illustrates one potential embodiment of a user interface of the care provider dashboard 214. The user interface 1300 may be embodied as a web page, native application, or other interface provided by the care provider device 104 for interaction with the care exchange server 102. The user interface 1300 illustratively includes a business intelligence control 1302 that displays business intelligence related to the care provider, a market analysis control 1304 that activates a market analysis builder as shown in FIG. 15 and described below, and an edit profile control 1306 that allows the care provider to create and/or update the associated provider profile. The user interface 1300 further includes a request status control 1308, which displays information for pending care coordinator requests organized by status. An accepting list 1310 displays requests that are currently accepting interest from matching care providers and thus may be accepted by the care provider. A reviewing list 1312 displays requests that the care provider has expressed interest in and that are being reviewed by the care coordinator and/or the individual patient. A matched list 1314 displays requests that have been accepted (matched) by the care coordinator and/or the individual patient. Each entry in the request status control 1308 may include summary information (e.g., patient name), and when selected may provide additional details as shown in FIG. 14, described below.

Note that in the illustrative embodiment, the accepting list 1310 includes a request for “individual 1,” while the accepting list 1006 of FIG. 10 includes requests for both “individual 1” and “individual 2.” Thus, in the illustrative example, the request for “individual 2” is not included in the accepting list 1310, for example because the active care provider is not eligible for the request from individual 2 (e.g., without matching service, geographic area, or funding source). The reviewing list 1312 includes requests for “individual 3” and “individual 4,” and the reviewing list 1008 of FIG. 10 includes requests for those individuals as well as “individual 5.” In that example, the request for individual 5 may have been responded to by a different care provider and thus is not visible in the care provider dashboard 214 of FIG. 13. Similarly, the matched list 1314 includes requests for “individual 6” and “individual 7,” while the matched list 1010 of FIG. 10 includes only a request for “individual 6.” In that example, the request for individual 7 may have originated from a different care coordinator, and thus is not visible in the care coordinator dashboard 216 of FIG. 10.

Referring now to FIG. 14, user interface 1400 illustrates one potential embodiment of a coordinator request detail view user interface of the care provider dashboard 214. The user interface 1400 may be embodied as a web page, native application, or other interface provided by the care provider device 104 for interaction with the care exchange server 102. The user interface 1400 includes a request detail control 1402, which displays care attributes of a selected care coordinator request, including the request timeframe. The illustrative request detail control 1402 also includes a control to extend the request timeframe (e.g., if the care provider requires additional time to express interest). A request status control 1404 indicates the status of the selected request, which is illustratively “reviewing” (i.e., being reviewed by the care coordinator and/or the individual patient). A coordinator control 1406 identifies the care coordinator that originated the care coordinator request. A messaging control 1408 allows the care provider to exchange messages with the care coordinator.

Referring now to FIG. 15, user interface 1500 illustrates one potential embodiment of a market analysis user interface of the care provider dashboard 214. The user interface 1500 may be embodied as a web page, native application, or other interface provided by the care provider device 104 for interaction with the care exchange server 102. The user interface 1500 includes a market analysis builder 1502, which may be used to configure attributes of a market analysis request. The user interface 1500 further includes graphical controls 1504, 1506 that display results of the market analysis. The graphical control 1504 is illustratively a line graph, for example to display care requests, matched requests, or other data over time. The graphical control 1506 is illustratively a heat map, for example to display care requests, matched requests, or other data in specified geographic areas. The geographic areas may be neighborhoods, postal codes (e.g., ZIP codes), census tracts, political boundaries, or other geographical areas. The user interface 1500 may also include other graphical or non-graphical controls to display the market analysis data, such as a pie charts, bar charts, tables, or other displays.

1. A computing device for processing care requests, the computing device comprising:
a care provider manager to register a plurality of care providers;
a care coordinator dashboard to receive a care coordinator request indicative of one or more care attributes from a care coordinator;
a data engine to select a first set of the plurality of care providers matching the one or more care attributes; and
a care provider dashboard to (i) send the care coordinator request to the first set of the plurality of care providers, and (ii) receive an interest response from a second subset of the first set, wherein the interest response is indicative of interest in the care coordinator request;
wherein the care coordinator dashboard is further to (i) provide the second subset of the plurality of care providers to the care coordinator, and (ii) receive an acceptance response from the care coordinator in response to provision of the second subset, wherein the acceptance response is indicative of an accepted care provider of the second subset.

2. The computing device of claim 1, wherein the data engine is further to update a care provider reliability score associated with the accepted care provider in response to receiving the acceptance response.

3. The computing device of claim 2, wherein to provide the second subset of the plurality of care providers to the care coordinator comprises to provide a provider profile associated with each of the second subset, wherein the provider profile comprises the reliability score.

4. The computing device of claim 2, wherein to update the care provider reliability score comprises to receive a reliability score from the care coordinator.

5. The computing device of claim 4, wherein to update the care provider reliability score further comprises to receive a reliability score from an individual patient associated with the care coordinator request.

6. The computing device of claim 1, wherein the care attributes comprises a requested service, a requested location, and a requested funding source.

7. The computing device of claim 1, wherein to provide the second subset of the plurality of care providers to the care coordinator comprises to send a personalized exchange viewer to an individual patient associated with the care coordinator request.

8. The computing device of claim 1, wherein:
the care coordinator dashboard is further to receive a negative acceptance response from the care coordinator in response to the provision of the second subset, wherein the negative acceptance response is indicative of a declined care provider of the second subset; and
the care provider dashboard is further to send a notification to a third subset of the second subset in response to receipt of the negative acceptance response, wherein the third subset does not include the declined care provider.

9. The computing device of claim 1, further comprising a business intelligence engine to:
generate business intelligence data for a first care provider based on care provider data associated with the first care provider; and
provide the business intelligence data to the first care provider.

10. The computing device of claim 9, wherein the business intelligence data comprises available care coordinator requests for a defined time period, reviewed care coordinator requests for the defined time period, or matched care coordinator requests for the defined time period.

11. The computing device of claim 1, further comprising a market analysis engine to:
receive a market analysis request including first care attributes from a first care provider;
select care coordinator data that matches the first care attributes of the market analysis request; and
provide the care coordinator data that matches the first care attributes to the first care provider.

12. The computing device of claim 11, wherein the first care attributes comprises a care coordinator identity, a service offered, a funding source, or a service geography.

13. The computing device of claim 1, wherein to register the plurality of care providers comprises, for each care provider, to configure a care provider profile, wherein the care provider profile comprises services offered, service geography, or funding sources accepted.

14. The computing device of claim 1, further comprising a web portal that comprises the care coordinator dashboard and the care provider dashboard.

15. A method for processing care requests, the method comprising:
registering, by a care exchange server, a plurality of care providers;
receiving, by the care exchange server, a care coordinator request indicative of one or more care attributes from a care coordinator;
selecting, by the care exchange server, a first set of the plurality of care providers matching the one or more care attributes;
sending, by the care exchange server, the care coordinator request to the first set of the plurality of care providers;
receiving, by the care exchange server, an interest response from a second subset of the first set, wherein the interest response is indicative of interest in the care coordinator request;
providing, by the care exchange server, the second subset of the plurality of care providers to the care coordinator; and
receiving, by the care exchange server, an acceptance response from the care coordinator in response to providing the second subset, wherein the acceptance response is indicative of an accepted care provider of the second subset.

16. The method of claim 15, further comprising updating, by the care exchange server, a care provider reliability score associated with the accepted care provider in response to receiving the acceptance response.

17. The method of claim 15, wherein the care attributes comprises a requested service, a requested location, and a requested funding source.

18. The method of claim 15, wherein providing the second subset of the plurality of care providers to the care coordinator comprises sending a personalized exchange viewer to an individual patient associated with the care coordinator request.

19. The method of claim 15, further comprising:
generating, by the care exchange server, business intelligence data for a first care provider based on care provider data associated with the first care provider; and
providing, by the care exchange server, the business intelligence data to the first care provider.

20. The method of claim 15, further comprising:
receiving, by the care exchange server, a market analysis request including first care attributes from a first care provider;
selecting, by care exchange server, care coordinator data that matches the first care attributes of the market analysis request; and
providing, by the care exchange server, the care coordinator data that matches the first care attributes to the first care provider.
